(19) United States
(12) Patent Application Publication
(10) Pub. No.: US 2023/0061393 A1
(43) Pub. Date: Mar. 2, 2023

(54) MASS AND FORCE INDICATORS IN DIAGNOSTICS AND OUTPUT CORRECTION FOR ELECTROCHEMICAL GAS SENSORS

(71) Applicant: Carrier Corporation, Palm Beach Gardens, FL (US)

(72) Inventors: Jon K. Evju, Shakopee, MN (US); Robert L. Fillmore, Bloomington, MN (US)

(21) Appl. No.: 17/898,211

(22) Filed: Aug. 29, 2022

Related U.S. Application Data
(60) Provisional application No. 63/239,595, filed on Sep. 1, 2021.

(51) Int. Cl. G01N 27/416; G01G 19/18

(52) U.S. Cl. CPC G01N 27/4163; G01G 19/18; G01N 27/4162

(57) ABSTRACT

A method of using mass and force indicators in electrochemical gas sensor management is provided. The method includes measuring a change in mass of an electrochemical gas sensor and determining a status of the electrochemical gas sensor based on results of the measuring of the change in mass.

### CROSS-REFERENCE TO RELATED APPLICATIONS

This application claims priority to U.S. Provisional Application Ser. No. 63/239,595, filed Sep. 1, 2021, the contents of which are hereby incorporated by reference in its entirety.

### BACKGROUND

The present disclosure relates to gas detectors and, more particularly, to mass and force indicators in diagnostics and output correction for electrochemical gas sensors in gas detectors.

The precision and life-span of life-saving toxic gas electrochemical sensors are negatively impacted by long-term extreme environmental conditions. Loss and gain of solvent (often water) in the electrolyte solution (often sulfuric acid) can give rise to corresponding changes in sensitivity toward the target gas. For example, some electrochemical sensors will absorb water vapor in humid environments until they begin leaking/spilling sulfuric acid when the volume of electrolyte exceeds the available internal volume. In the other extreme (dry) case, the sulfuric acid electrolyte may become so concentrated that the acid attacks the materials and seals in the sensor housing, causing leaks and potentially catastrophic detector damage through corrosion. In the other extreme, the loss of electrolyte solution volume may expose the electrodes to air leading to sensor failure.

To assess the state of the sensor, voltage pulse, current pulse, and impedance spectroscopy are frequently employed; however, these techniques are indirect measurements that may fail to alert to all sensor failures (such as the extreme dry and/or leaking cases scenarios described above).

### BRIEF DESCRIPTION

According to an aspect of the disclosure, a method of using mass and force indicators in electrochemical gas sensor management is provided. The method includes measuring a change in mass of an electrochemical gas sensor and determining a status of the electrochemical gas sensor based on results of the measuring of the change in mass.

In accordance with additional or alternative embodiments, the measuring of the change in mass includes attaching a mass sensor to the electrochemical gas sensor and mounting the electrochemical gas sensor with the mass sensor in a manner that engages the mass sensor.

In accordance with additional or alternative embodiments, the mass sensor includes one or more of a strain gauge assembly, a piezo-electric element, an elastic element and a flexible circuit with a built-in or integral strain gauge.

In accordance with additional or alternative embodiments, the determining includes calibrating the electrochemical gas sensor prior to a usage thereof to determine an electrolyte sensitivity factor thereof, executing field sensitivity corrections using the results of the measuring of the change in mass to update the electrolyte sensitivity factor and calculating a field gas concentration based on the electrolyte sensitivity factor.

In accordance with additional or alternative embodiments, the calibrating includes factory calibration.

In accordance with additional or alternative embodiments, the calibrating includes calculating a response sensitivity factor of the electrochemical gas sensor, calculating an amount of an acid electrolyte in the electrochemical gas sensor and determining the electrolyte sensitivity factor from the response sensitivity factor and the amount of the acid electrolyte.

In accordance with additional or alternative embodiments, the executing of the field sensitivity corrections includes periodically executing the field sensitivity corrections.

In accordance with additional or alternative embodiments, the executing of the field sensitivity corrections includes determining whether the change in mass is within threshold limits and issuing a warning of sensor failure in an event the change in mass is not within the threshold limits.

In accordance with additional or alternative embodiments, the method further includes issuing a warning of sensor failure based on a result of the calculating of the field gas concentration.

According to an aspect of the disclosure, an electrochemical gas sensor assembly is provided and includes an electrochemical gas sensor, a mass sensor coupled to the electrochemical gas sensor and configured to measure a change in a mass of the electrochemical gas sensor over time and a processing system configured to determine a status of the electrochemical gas sensor based on the change in the mass.

In accordance with additional or alternative embodiments, the electrochemical gas sensor includes an enclosure including a body defining an interior and an opening by which the interior is communicative with an exterior environment, a membrane disposed on an interior side of the opening and a filter disposed on an exterior side of the opening, a printed circuit board (PCB) affixed to an exterior of the body, electrodes operably disposed in the interior and coupled with the PCB and an electrolyte filling at least a portion of the interior around the electrodes.

In accordance with additional or alternative embodiments, the electrolyte includes an aqueous or non-aqueous fluid.

In accordance with additional or alternative embodiments, the aqueous or non-aqueous fluid is replaceable by water in a humid environment via at least one of: the filter, the opening, and the membrane and the aqueous or non-aqueous fluid evaporates out of the interior in a dry environment via at least one of: the membrane, the opening, and the filter.

In accordance with additional or alternative embodiments, the mass sensor is attached to the PCB.

In accordance with additional or alternative embodiments, the electrochemical gas sensor is suspended on the mass sensor.

In accordance with additional or alternative embodiments, the mass sensor includes a deflectable member and a strain gauge.

In accordance with additional or alternative embodiments, the mass sensor includes a piezoelectric element.

In accordance with additional or alternative embodiments, the mass sensor includes an elastic element and a grid for optical reading.

In accordance with additional or alternative embodiments, the processing system issues a warning in at least one of an event the change in the mass exceeds threshold limits and a mass corrected sensitivity of the electrochemical gas sensor decreases beyond a predefined threshold based on the change in the mass.

According to an aspect of the disclosure, an electrochemical gas sensor assembly is provided and includes a flexible circuit and an electrochemical gas sensor suspended on the flexible circuit and including an enclosure that includes a body defining an interior into which the flexible circuit extends and an opening by which the interior is communicative with an exterior environment, a membrane disposed on an interior side of the opening and a filter disposed on an exterior side of the opening, electrodes operably disposed in the interior and coupled with contact pads of the PCB and an electrolyte filling at least a portion of the interior around the electrodes. The electrochemical gas sensor further includes a mass sensor built-in or integral with the flexible circuit and configured to measure a change in a mass of the electrochemical gas sensor over time and a processing system configured to determine a status of the electrochemical gas sensor based on the change in the mass.

Additional features and advantages are realized through the techniques of the present disclosure. Other embodiments and aspects of the disclosure are described in detail herein and are considered a part of the claimed technical concept. For a better understanding of the disclosure with the advantages and the features, refer to the description and to the drawings.

### BRIEF DESCRIPTION OF THE DRAWINGS

For a more complete understanding of this disclosure, reference is now made to the following brief description, taken in connection with the accompanying drawings and detailed description, wherein like reference numerals represent like parts:

FIG. 1 is a flow diagram illustrating a method of using mass and force indicators in electrochemical gas sensor management in accordance with embodiments;

FIG. 2 is a graphical flow diagram illustrating details of the method of FIG. 1 in accordance with further embodiments;

FIG. 3 is a schematic side view of an exemplary electrochemical gas sensor assembly in accordance with embodiments;

FIG. 4 is a graphical depiction of a relationship between a mass of an electrochemical gas sensor and a mass corrected sensitivity of the electrochemical gas sensor in accordance with embodiments;

FIG. 5 is a schematic side view of an exemplary electrochemical gas sensor assembly in accordance with embodiments;

FIG. 6 is a schematic side view of an exemplary electrochemical gas sensor assembly in accordance with embodiments; and

FIG. 7 is a schematic side view of an exemplary electrochemical gas sensor assembly in accordance with embodiments.

### DETAILED DESCRIPTION

In some, less extreme cases, sensor mass changes are used to correct for the changes in sensor sensitivity to target gases as the electrolyte solution gets more or less concentrated through evaporation or absorption of moisture. These mechanisms affect the viscosity, ion mobility and gas solubility of the electrolyte solution inside the electrochemical gas sensor, which affect sensor sensitivities to target gas.

As will be described below, losses or gains of water/solvent are directly evident as mass gains or losses that correlate directly with sensitivity changes. This correlation holds over a wide range for the sensors' sensitivity. With today's light-weight polymer sensor housings, the respective change in mass can represent 15-40% of the total sensor mass and is readily measured. Combining this significant mass change with modern methods suitable for measuring the force of an object due to gravity working on it, it becomes easy to implement a direct diagnostics tool. Over most of the useful range, the direct diagnostics tool can be implemented completely without perturbing the electrochemical sensor. Toward the end of the useful life, in an environment where the electrolyte is drying out, it may become advantageous to implement additional diagnostic tools or thresholds which are well known in the prior art. The output from any or all of these diagnostic methods may be employed to warn the user of nearing sensor failure to allow replacement of sensor, or sensor assembly, minimizing downtime.

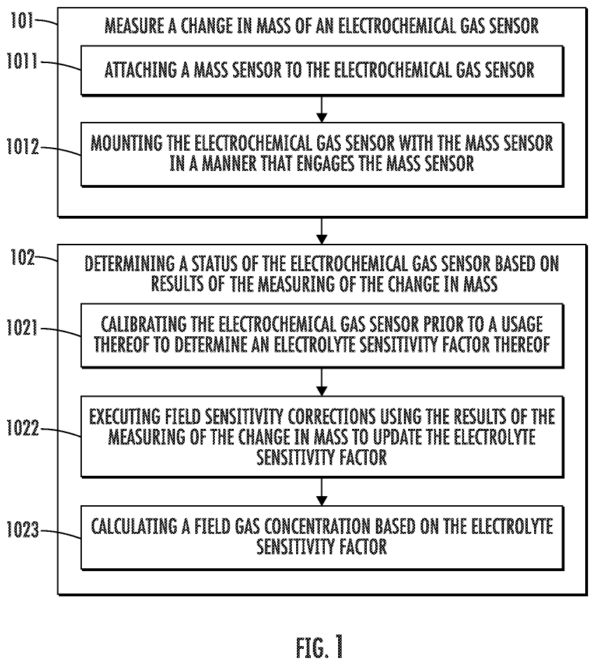

With reference to FIG. 1, a method of using mass and force indicators in electrochemical gas sensor management is provided. The method includes measuring a change in mass of an electrochemical gas sensor (101) and determining a status of the electrochemical gas sensor based on results of the measuring of the change in mass (102). The measuring of the change in mass of operation 101 can include attaching a mass sensor to the electrochemical gas sensor (1011), mounting the electrochemical gas sensor with the mass sensor in a manner that engages the mass sensor (1012) and measuring a mass of the unused or new electrochemical gas sensor (1013). In accordance with embodiments, the mass sensor can include one or more of a strain gauge assembly, a piezo-electric element, and/or an elastic element. The determining of operation 102 can include calibrating (i.e., at the factory or otherwise prior to electrochemical gas sensor usage) the electrochemical gas sensor prior to a usage thereof to determine an electrolyte sensitivity factor thereof (1021), executing field sensitivity corrections (i.e., at predefined times, intervals or periodically) using the results of the measuring of the change in mass to update the electrolyte sensitivity factor (1022) and calculating a field gas concentration based on the electrolyte sensitivity factor (1023).

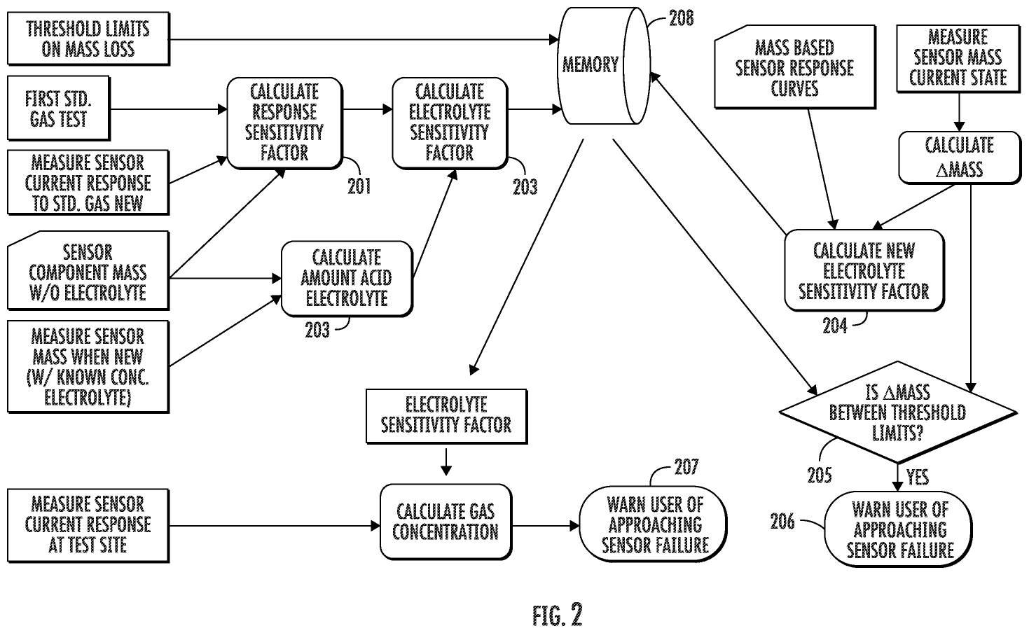

With reference to FIG. 2, further embodiments of the method of FIG. 1 are illustrated. For example, as shown, the calibrating of operation 1021 can include calculating a response sensitivity factor of the electrochemical gas sensor (201) from a sensor mass without electrolyte, from a standard gas test, and from a measurement of a sensor current response during the standard gas test. Also, the calibrating of operation 1021 can include calculating an amount of an acid electrolyte in the electrochemical gas sensor (202) from the sensor mass and the sensor mass with electrolyte at the factory or prior to usage. In addition, the calibrating of operation 1021 can include determining the electrolyte sensitivity factor from the response sensitivity factor and the amount and concentration of the acid electrolyte (203) per the measured sensor mass changes

As another example, the executing of the field sensitivity corrections using the results of the measuring of the change in mass to update the electrolyte sensitivity factor of operation 1022 can include calculating a new electrolyte sensitivity factor from the change in mass and from known mass-based sensor response curves (204). Also, the executing of the field sensitivity corrections using the results of the measuring of the change in mass to update the electrolyte sensitivity factor of operation 1022 can include determining whether the change in mass is within threshold limits (205) or, if it is in agreement with results from standard electrochemical characterization methods, recent calibration events or historical environmental factors (Temperature, humidity and pressure) and issuing a warning of sensor failure in an event the change in mass is not within the threshold limits (206).

As another example, the method can further include issuing a warning of sensor failure based on a result of the calculating of the field gas concentration (207), which takes into account the electrolyte sensitivity factor (original or updated/new) and measured sensor current response at a test site, and inputting into a memory (208) of a processing system 330 (see FIGS. 3-5) threshold limits on mass changes and original and updated/new electrolyte sensitivity factors.

With reference to FIGS. 3-6, an electrochemical gas sensor assembly 301 is provided and includes an electrochemical gas sensor 310, a mass sensor 320, and the processing system 330. The mass sensor 320 is coupled to the electrochemical gas sensor 310 and is configured to measure a change in a mass of the electrochemical gas sensor 310 over time. The processing system 330 includes the memory 208 of FIG. 2 and is configured to determine a status of the electrochemical gas sensor based on the change in the mass read by the mass sensor 320.

The electrochemical gas sensor assembly 310 includes an enclosure 311, an optional printed circuit board (PCB) 312, electrodes 313, and an electrolyte 314. The enclosure 311 includes a body 315 that is formed to define an interior 316 and one or more capillary openings (hereinafter referred to as an “opening”) 317 by which the interior 316 is communicative with an exterior environment. The enclosure 311 may further include a membrane 318, which is disposed on an interior side of the opening 317, and a filter (i.e., a capillary filter) 319, which is disposed on an exterior side of the opening 317. The PCB 312 is affixed to an exterior of the body 315 and includes the memory 208 of FIG. 2 and a processor which is configured to read and to execute executable instructions stored in the memory 208 in order to execute the method(s) described herein. The electrodes 313 are operably disposed in the interior 316 and are operably coupled with the PCB 312. The electrodes 313 can include a counter electrode 3131, a reference electrode 3132 and a sensing (working) electrode 3133. The electrolyte 314 fills at least a portion of the interior 316 around the electrodes 313 and can include or be provided as an aqueous or non-aqueous fluid, such as an aqueous acid electrolyte or another similar electrolyte.

In accordance with embodiments, the electrolyte 314 can be diluted over time by water in a humid environment entering the sensor via at least one of: the filter 319, the opening 317, and the membrane 318. This increases the mass of the electrochemical gas sensor 310. Conversely, some quantity of water can evaporate out of the interior 316 in a dry environment from the electrolyte 314 via at least one of: the membrane 318, the opening 317, and the filter 319. This can decrease a mass of the electrochemical gas sensor 310.

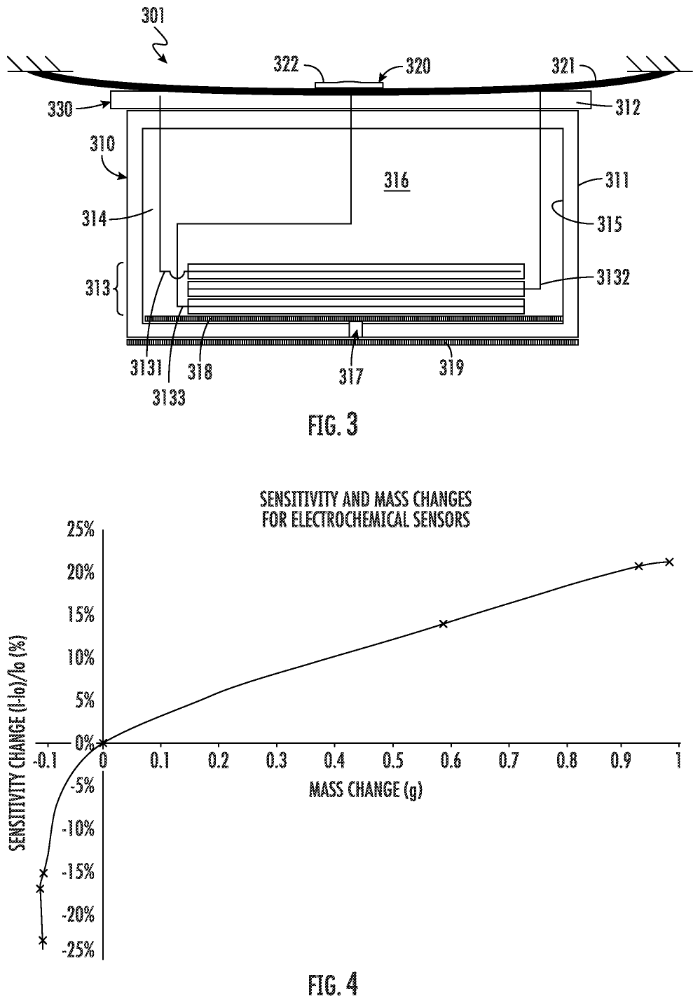

As shown in FIG. 4, the increase or decrease in the mass of the electrochemical gas sensor 310 correlates with a sensitivity change in the electrochemical gas sensor 310. This sensitivity change needs to be taken into account when readings of the electrochemical gas sensor 310 are taken in terms of reliability. Also, the sensitivity change can be considered in terms of making a decision about issuing a warning as to the status of the electrochemical gas sensor 310 and/or as to making a decision about replacing the electrochemical gas sensor 310. As such, the ability to determine the change in the mass of the electrochemical gas sensor 310, which is provided by the mass sensor 320, is a valuable advance as compared to techniques that never considered the change in mass as being indicative of changes to gas sensor response.

The processing system 330 can be configured to issue a warning in at least one of an event the change in the mass exceeds threshold limits and a mass corrected sensitivity of the electrochemical gas sensor 310 decreases beyond a predefined threshold based on the change in the mass.

With continued reference to at least FIGS. 3, 5 and 6, the mass sensor 320 can be attached to the PCB 312 with the electrochemical gas sensor 310 as a whole being effectively suspended on the mass sensor 320.

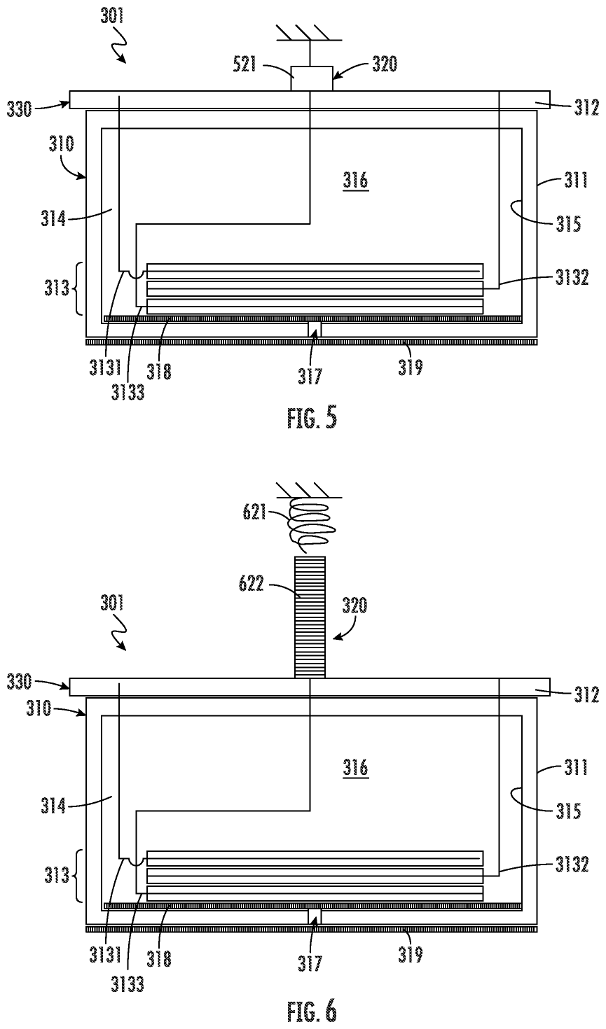

That is, as shown in FIG. 3, the mass sensor 320 can include a deflectable member 321, which can be fastened at opposite ends thereof to a roof or ceiling and affixed at a center thereof to the PCB 312, and a strain gauge 322. In this case, the deflectable member 321 deflects upwardly or downwardly based on the change in mass of the electrochemical gas sensor 310. The strain gauge 322 is capable of detecting the strain associated with this deflection and thus effectively sensing the change in the mass of the electrochemical gas sensor 310. As shown in FIG. 5, the mass sensor 320 can include a piezoelectric element 521, which can be fastened to a roof or ceiling and affixed to the PCB 312. In this case, the piezoelectric element 521 generates a current based on the change in mass of the electrochemical gas sensor 310. This current can be used to effectively sense the change in the mass of the electrochemical gas sensor 310. As shown in FIG. 6, the mass sensor 320 can include an elastic element 621 and a grid for optical reading 622 which operate similarly as the deflectable member 321 and the strain gauge 322 of FIG. 3.

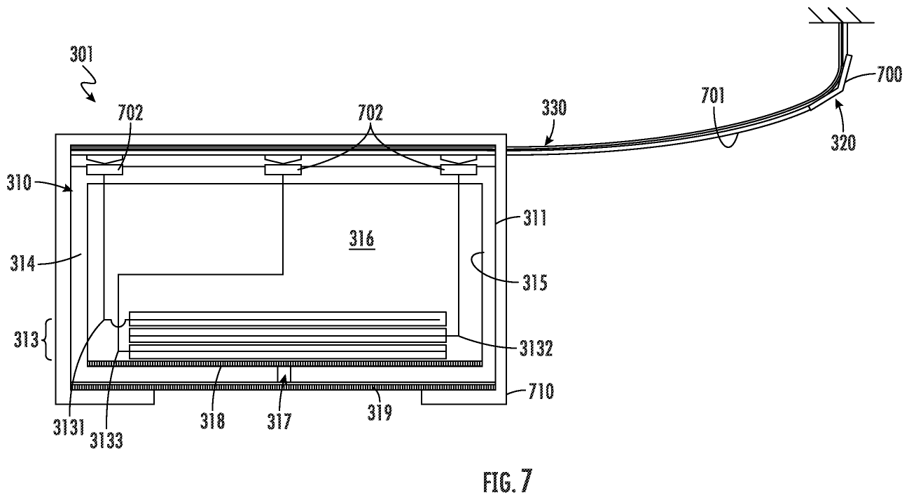

With reference to FIG. 7, the mass sensor 320 can be provided as a strain gauge 700 or another similar device and can be built-into or integral with a flexible circuit 701 that extends into the interior 316 of the body 315. In these or other cases, the electrodes 313 can be coupled with the flexible circuit 701 via contact pads 702 of the flexible circuit 701 and the processing system 330 can be incorporated into the flexible circuit 701. As shown in FIG. 7, a sensor clip or housing 710 can be provided to secure the electrochemical gas sensor 310 to the flexible circuit 701 and to ensure that there is good and reliable electrical connections between the electrodes 313 and the contact pads 702.

It should be appreciated that the mass sensor 320 may be any electrical or mechanical device capable of measuring a mass change, and, may be positioned on any suitable location capable of providing an accurate indication of a change in mass of the electrochemical gas sensor 310. It is to be understood that additional features, such as accelerometers, gyroscopes and other similar components, can be incorporated either in the sensor assembly or in the detector itself. In any case, these additional features can serve to collect data on alignment of the gas sensor (i.e., in a case of non-vertical alignment in mounting) as well as vibrations (i.e., in a case in which gas detector is mounted in an area of high, expected vibration) in order to correct gravitational measurements accordingly.

Technical effects and benefits of the present disclosure are the provision of systems and methods of using mass and force indicators in diagnostics and output corrections for electrochemical gas sensors in gas detectors. Since the mass and force changes translate to changes in concentration and volume of electrolyte, it is possible to assess the electrochemical sensors' sensitivity and to adjust detector output accordingly and to determine if the sensor is approaching or passing a threshold where a replacement should be made. It is also relatively easy to set thresholds to trigger sensor replacements before the sensors begin leaking or before the sensor dries out to expose an electrode to air.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the technical concepts in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

While the preferred embodiments to the disclosure have been described, it will be understood that those skilled in the art, both now and in the future, may make various improvements and enhancements which fall within the scope of the claims which follow. These claims should be construed to maintain the proper protection for the disclosure first described.

What is claimed is:

1. A method of using mass and force indicators in electrochemical gas sensor management, the method comprising:
measuring a change in mass of an electrochemical gas sensor; and
determining a status of the electrochemical gas sensor based on results of the measuring of the change in mass.

2. The method according to claim 1, wherein the measuring of the change in mass comprises:
attaching a mass sensor to the electrochemical gas sensor; and
mounting the electrochemical gas sensor with the mass sensor in a manner that engages the mass sensor.

3. The method according to claim 1, wherein the mass sensor comprises one or more of a strain gauge assembly, a piezo-electric element, an elastic element, and a flexible circuit with a built-in or integral strain gauge.

4. The method according to claim 1, wherein the determining comprises:
calibrating the electrochemical gas sensor prior to a usage thereof to determine an electrolyte sensitivity factor thereof;
executing field sensitivity corrections using the results of the measuring of the change in mass to update the electrolyte sensitivity factor; and
calculating a field gas concentration based on the electrolyte sensitivity factor.

5. The method according to claim 4, wherein the calibrating comprises factory calibration.

6. The method according to claim 4, wherein the calibrating comprises:
calculating a response sensitivity factor of the electrochemical gas sensor;
calculating an amount of an acid electrolyte in the electrochemical gas sensor; and
determining the electrolyte sensitivity factor from the response sensitivity factor and the amount of the acid electrolyte.

7. The method according to claim 4, wherein the executing of the field sensitivity corrections comprises periodically executing the field sensitivity corrections.

8. The method according to claim 4, wherein the executing of the field sensitivity corrections comprises:
determining whether the change in mass is within threshold limits; and
issuing a warning of sensor failure in an event the change in mass is not within the threshold limits.

9. The method according to claim 4, further comprising issuing a warning of sensor failure based on a result of the calculating of the field gas concentration.

10. An electrochemical gas sensor assembly, comprising:
an electrochemical gas sensor;
a mass sensor coupled to the electrochemical gas sensor and configured to measure a change in a mass of the electrochemical gas sensor over time; and
a processing system configured to determine a status of the electrochemical gas sensor based on the change in the mass.

11. The electrochemical gas sensor assembly according to claim 10, wherein the electrochemical gas sensor comprises:
an enclosure comprising a body defining an interior and an opening by which the interior is communicative with an exterior environment, a membrane disposed on an interior side of the opening and a filter disposed on an exterior side of the opening;
a printed circuit board (PCB) affixed to an exterior of the body;
electrodes operably disposed in the interior and coupled with the PCB; and
an electrolyte filling at least a portion of the interior around the electrodes.

12. The electrochemical gas sensor assembly according to claim 11, wherein the electrolyte comprises an aqueous or non-aqueous fluid.

13. The electrochemical gas sensor assembly according to claim 12, wherein:
the aqueous or non-aqueous fluid is replaceable by water in a humid environment via at least one of: the filter, the opening, and the membrane, and
the aqueous or non-aqueous fluid evaporates out of the interior in a dry environment via at least one of: the membrane, the opening, and the filter.

14. The electrochemical gas sensor assembly according to claim 11, wherein the mass sensor is attached to the PCB.

15. The electrochemical gas sensor assembly according to claim 11, wherein the electrochemical gas sensor is suspended on the sensor.

16. The electrochemical gas sensor assembly according to claim 10, wherein the mass sensor comprises a deflectable member and a strain gauge.

17. The electrochemical gas sensor assembly according to claim 10, wherein the mass sensor comprises a piezoelectric element.

18. The electrochemical gas sensor assembly according to claim 10, wherein the mass sensor comprises an elastic element and a grid for optical reading.

19. The electrochemical gas sensor assembly according to claim 10, wherein the processing system issues a warning in at least one of:
an event the change in the mass exceeds threshold limits, and
a mass corrected sensitivity of the electrochemical gas sensor decreases beyond a predefined threshold based on the change in the mass.

20. An electrochemical gas sensor assembly, comprising:
a flexible circuit;
an electrochemical gas sensor suspended on the flexible circuit and comprising an enclosure comprising a body defining an interior into which the flexible circuit extends and an opening by which the interior is communicative with an exterior environment, a membrane disposed on an interior side of the opening and a filter disposed on an exterior side of the opening, electrodes operably disposed in the interior and coupled with contact pads of the PCB and an electrolyte filling at least a portion of the interior around the electrodes;
a mass sensor built-in or integral with the flexible circuit and configured to measure a change in a mass of the electrochemical gas sensor over time; and
a processing system configured to determine a status of the electrochemical gas sensor based on the change in the mass.
